(19) United States
(12) Patent Application Publication
(10) Pub. No.: US 2012/0321666 A1
(43) Pub. Date: Dec. 20, 2012

(54) T CELL THERAPY FOR B CELL LYMPHOMA

(76) Inventors: Laurence J. N. Cooper, Houston, TX (US); Heather Wilson-Robles, College Station, TX (US); Colleen M. O'Connor, Houston, TX (US); Cassie A. Hartline, Houston, TX (US); Sabina Sheppard, College Station, TX (US)

(21) Appl. No.: 13/479,163

(22) Filed: May 23, 2012

Related U.S. Application Data
(60) Provisional application No. 61/489,176, filed on May 23, 2011.

(51) Int. Cl. A61K 35/12; A61P 37/04; A61P 35/00; C12N 5/0783; A61K 35/14

(52) U.S. Cl. 424/278.1; 435/350

(57) ABSTRACT

Disclosed are methods and compositions for improving anti-tumor response and survivability in patients with cancer, such as non-Hodgkin lymphoma. In certain aspects, methods are provided for infusing lymphoma patients with T cells that are propagated ex vivo. Also provided are methods and compositions for propagating canine T cells ex vivo for infusion into cancer patients.

### REFERENCE TO RELATED APPLICATIONS

This application claims the benefit of U.S. provisional application No. 61/489,176 filed May 23, 2011 and is herein incorporated by reference in its entirety.

### BACKGROUND OF THE INVENTION

I. Field of the Invention

The present invention relates generally to the field of oncology. More specifically, the invention relates to methods and compositions for improving tumor response and propagating T-cells ex vivo.

II. Related Art

Lymphomas are cancers of the white blood cells, lyphocytes. Cancer in canines models human malignancies due to canine large body size, genetic similarity, spontaneous occurrence of a broad diversity of tumors, and similar treatment modalities. The etiology of spontaneous canine and human cancers is analogous as both may be induced through genetic abnormalities or predisposition and common environmental exposures.

### SUMMARY OF THE INVENTION

In one aspect, the present invention provides a method of providing an anti-tumor response in a mammalian subject, such as a canine, with cancer comprising infusing the subject with T cells. In one embodiment, the T cells of the invention are autologous and in another embodiment they are ex-vivo propagated prior to infusing. In another embodiment, the T cells are propagated by culturing peripheral blood mononuclear cells with γ-irradiated artificial antigen presenting cells and a cytokine. In yet another embodiment, the artificial antigen presenting cells are loaded with a CD3 antibody, such as OKT3. In a certain embodiment the peripheral blood mononuclear cells are canine cells. In particular embodiments the cytokine is IL-2 or IL-21 and in one embodiment may comprise both IL-2 and IL-21.

In yet another embodiment, the T cells of the invention comprise at least one marker selected from the group consisting of CD3⁺, CD4⁺, CD8⁺, CD25⁺, CD56⁺, CD21⁺ and CCR7⁺ and in particular embodiments are CD3⁺CD8⁺ cells or CD3⁺CD4⁺ cells. In a further embodiment, infusion of the T cells is intravenous.

In another embodiment, the subject receives chemotherapy as part of a treatment for the cancer, which can be contemporaneous to the infusion of T cells. For instance, the subject can receive chemotherapy prior to, during or after infusion of the T cells. In particular embodiments the chemotherapy comprises cyclophosphamide, hydroxydaunorubicin (doxorubicin), Oncovin (vincristine), and prednisone/prednisolone, which combination is also known as CHOP. In certain embodiments, the T cells are infused about 7 to about 488 days after completion of chemotherapy, for instance, the T cells can be infused about 4, 5, 6, 7, 8, 9 or 10 to about 18, 19, 20, 21, 22, 23 or 24 days, about 95, 96, 97, 98, 99, 100 or 101 to about 109, 110, 111, 112, 113, 114 or 115 days or about 473, 474, 475, 476, 477, 478 or 479 to about 485, 486, 487, 488, 489, 490 or 491 days after the completion of chemotherapy. In another embodiment, the subject is infused with about 5×10⁷/m² to about 3×10⁹ cells/m² T cells, for instance, about 3×10⁷/m², 4×10⁷/m², 5×10⁷/m², 3×10⁸/m², 4×10⁸/m², 5×10⁸/m², 3×10⁹/m², 4×10⁹/m², 5×10⁹/m².

In a further embodiment, the subject is a human or a canine. In yet a further embodiment, the subject has cancer selected from the group consisting of non-Hodgkin lymphoma, Burkitt's lymphoma, leukemia, hematological cancer, colorectal cancer, solid tumor, bone cancer, lung cancer, brain tumor, glioma, heart cancer, skin cancer, melanoma, basal cell carcinoma, renal cell carcinoma, liver cancer, thyroid cancer, breast cancer, ovarian cancer, cervical cancer, endometrial cancer, prostate cancer, testicular cancer, throat cancer, esophageal cancer, gastric cancer, oral cancer or pancreatic cancer.

In another aspect, the invention provides a method for propagating canine T cells comprising culturing canine peripheral blood mononuclear cells with γ-irradiated artificial antigen presenting cells and a cytokine. In one embodiment the artificial antigen presenting cells are genetically modified to express T cell co-stimulatory ligands, for instance, the T cell co-stimulatory ligands may be selected from the group consisting of CD19, CD64, CD86, CD137L, and membrane bound IL-15. In another embodiment the artificial antigen presenting cells are loaded with a CD3 antibody, such as OKT3. In certain embodiments, the cytokine is an exogenous interleukin, such as IL-2 or IL-21, and in one embodiment the cytokine may be IL-2 and IL-21

In yet another embodiment, the canine peripheral blood mononuclear cells are isolated from a canine with non-Hodgkin lymphoma. In still another embodiment, the peripheral blood mononuclear cells are cultured with γ-irradiated artificial antigen presenting cells and a cytokine for up to 35 days, for instance about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 days. In a further embodiment, the T cells comprise at least one marker selected from the group consisting of CD3⁺, CD4⁺, CD8⁺, CD25⁺, CD56⁺, CD21⁺ and CCR7⁺ and in particular embodiments are CD3⁺CD8⁺ cells or CD3⁺CD4⁺ cells.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows the composition of circulating canine T cells. The expression of T-cell subsets in PB from (a) healthy subjects (n=4) and (b) canines with NHL (n=4) prior to CHOP treatment. Percentage expression is based on cells within a lymphocyte gate described by a Forward vs. Side Scatter flow cytometry plot. Mean±s.e.m. are shown.

FIG. 2 (a) demonstrates that rhIL-21 preferentially propagates canine CD8⁺ T cells on OKT3-loaded aAPC. T cells isolated from PBMC of healthy dogs were numerically expanded on γ-irradiated OKT3-loaded aAPC at a T-cell:aAPC ratio of 2:1. T cells were re-stimulated with aAPC every 7 days. Recombinant human IL-21 and/or rhIL-2 were added to the co-culture of T cells on aAPC as shown. Parental K562 cells were genetically modified to co-express human CD64 and the human T-cell co-stimulatory molecules CD86, CD137L, and membrane-bound IL-15 (co-expressed with EGFP) and cloned (CLN4). OKT3 was loaded via CD64 on CLN4 and detected with a Fab-specific antibody. CLN4 is shown as gray-open histograms and K562 parental cells as black-open histograms.

FIGS. 2 (b) and (c) demonstrate (b) numeric expansion of T cells on γ-irradiated OKT3-loaded aAPC (added every 7 days) in the presence of rhIL-2, or both rhIL-2 and rhIL-21. The average cell counts from two donors are shown. (c) The mean viability of T cells during co-culture on aAPC and cytokine(s) (n=2).

FIG. 2 (d)-(f) shows percentage expression (n=2) of (d) CD3⁺CD8⁺ and (e) CD3⁺CD4⁺ T cells upon co-culture with aAPC and cytokines(s). (f) Numeric expansion of T cells measured over culture time on aAPC and rhIL-2 and 21 recorded as the mean inferred cell count (black dashed line) based on 5 healthy subjects and 5 non-trial canines with NHL. Upward arrows indicate timing for the addition of γ-irradiated OKT3-loaded aAPC.

FIGS. 3 (a) and (b) shows the characterization of T-cell infusion products. (a) The mean inferred cell count for 6 (of the 8) canines infused with T cells that were propagated over 28-35 days on γ-irradiated OKT3-loaded aAPC (CLN4) in presence of rhIL-2/IL-21. Arrows represent days aAPC were added. (b) Percent expression of CD4⁺ and CD8⁺ T-cell subsets during propagation on γ-irradiated aAPC in the presence of rhIL-2 μL-21. Arrows represent days aAPC were added.

FIGS. 3 (c) and (d) shows (c) immunophenotype of the T-cell infusion products (n=6) at the time of cryopreservation. The horizontal lines describe mean percentage expression. (d) T-cell infusion products were tested for IFN-γ production after stimulation with OKT3-loaded aAPC in the presence of rhIL-2/IL-21 (n=3). Analysis of multiplexed digital gene profiling of PB-derived canine T cells before and after propagation from healthy subjects (n=2) and canines with NHL (n=6) identified mRNA species which were significantly differentially expressed (p<0.001).

FIG. 3 (e) shows up-regulated (>2 fold) change in both healthy subjects and in at least 5 of 6 canines with NHL.

FIG. 3 (f) shows down-regulated (<2 fold) change in both healthy subjects and in at least 5 of 6 canines with NHL.

FIG. 3 (g) shows a study design: Enrollment occurred pre-, post, or during treatment with CHOP. Enrolled canines received one course of CHOP, typically administered over 19 weeks, and received one to three infusions of T cells 14 days apart using an intra-patient dose-escalation scheme.

FIG. 4 demonstrates the tracking of infused T cells. (a) Ratio of CD4:CD8 T cells in PB after CHOP, but before adoptive transfer of T cells and compared with measurements taken three hours after each T-cell infusion. The grey shaded area represents the mean CD4:CD8 ratio (1.6:1) in healthy subjects (n=4). (b) Mean T-cell counts in PB from 6 canines before and after adoptive transfer of T cells. The grey shaded area represents the range for CD8⁺ T cells in healthy subjects (n=4, 581 to 958 cells/μL). Arrows represent days T cells were infused. (c) Mean expression of CD3⁺ T cells pre-stained with red fluorescent dye, PKH-26, in the PB of 6 canines. Arrows represent days T cells were infused. (d) Evaluation of fluorescence and staining of CD3 from a LN biopsy. Frozen tissues were viewed with fluorescent microscopy to detect (top) PKH-26⁺ T cells and (bottom) co-stained with anti-CD3 to validate T-cell phenotype.

FIG. 5 demonstrates that adoptive transfer of ex vivo propagated T cells after CHOP improves survival of canines with NHL. Differences between 12 historical stage-, age-, and size-matched controls treated with CHOP (dashed line) and 7 (curve A) or 8 (curve B) research participants treated with CHOP and T cells (solid line) over 500 days in (a) tumor-free survival measured after achieving CR (p=0.005) and (b) overall survival measured upon diagnosis of NHL diagnosis (p=0.03). One dog (P42) had progressive disease at the time of T-cell infusions and was not evaluated in the tumor-free survival analysis, but was included in the overall survival analysis. This patient had stable disease for 112 days after study day 14.

FIG. 6 shows biomarkers that inform on T-cell engraftment and canine survival. (a) Average serum TK concentration in canines (n=5) plotted with the average number of CD3⁺CD8⁺ T cells in PB. (b) NLR on study day 35 plotted as a function of tumor-free survival. Arrow indicates NLR in PB of healthy canines. (c) Expression of granzyme B in propagated T cells, assessed at time of cryopreservation, plotted as a function of tumor-free survival.

FIG. 7 demonstrates TK concentrations for selected patients over the course of the study.

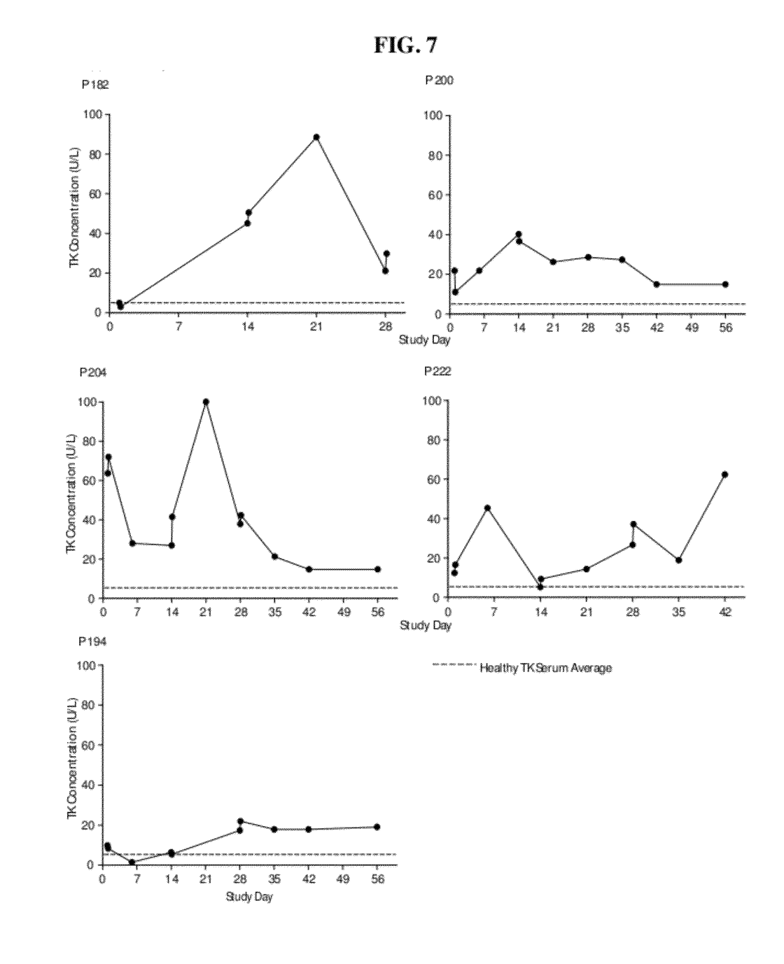

FIG. 8 shows average in vitro TK levels in conditioned supernatants. Conditioned tissue culture supernatants (triplicate) were collected after 48 hours to assess the impact of T cell proliferation and release of TK. TK levels were measured from γ-irradiated or non-irradiated CLN4 as 16.4±0.2 U/L and 12.1±0.5 U/L, respectively. The source of TK appears to be from proliferating T cells (n=2) as culturing T cells with cytokines alone increased TK concentration (6.0±0.8 U/L) compared to γ-irradiated T cells (0.9±0.4 U/L, p=0.01). Furthermore, co-culturing T cells with cytokines and γ-irradiated OKT3-loaded CLN4 elevated TK levels (28.6±7.5 U/L, p=0.04) compared to TK produced by T cells cultured with cytokines alone. These data imply that activated T cells produce TK at levels superior to non-activated T cells.

### DETAILED DESCRIPTION OF ILLUSTRATIVE EMBODIMENTS

The present invention provides methods and compositions for treating patients with cancer. For instance, the invention provides methods and compositions for treating patients with B cell lymphomas, such as non-Hodgkin lymphoma (NHL). In a particular aspect, the invention provides methods to generate T cells and infusion of T cells propagated ex vivo to improve anti-tumor response and survival of patients with cancer, such as lymphoma. Such methods, termed adoptive cellular therapy (ACT), generally involve the process of removing white blood cells (WBC) from a patient's blood or tumor, expanding and activating the WBC on an artificial system ex vivo to improve therapeutic potential and infusing the product back into the patient as a cancer treatment.

In one embodiment, the invention provides improved treatments over those known in the art. Cancers, such as lymphomas, can be treated by combinations of chemotherapy, immunotherapy, radiation and hematopoietic stem cell transplantation. For instance, the standard-of-care treatment for canine B-lineage NHL is the combination chemotherapy regimen of cyclophosphamide, vincristine, doxorubicin, and prednisone (CHOP). This induces a temporary remission in approximately 85% of canines, but is rarely curable and the two-year survival rate is less than 20%. Chemotherapy, using cytoreductive effects, can also modulate tumors and their microenvironment to present neo-antigens. However, the immune response to tumor-associated antigens (TAA) is compromised after chemotherapy due to iatrogenic lymphodepletion.

The T-cell therapy of the present invention targets malignant disease by employing mechanisms independent of chemo-radio-therapies. For instance, autologous ex vivo propagated T cells persist after infusion into a patient and retain trafficking molecules for homing to tumor sites. Furthermore, unlike antibodies, T cells can navigate to and through sites of bulky tumors using active motility mechanisms that allow them to enter into diseased tissue that have high interstitial pressures.

Another aspect of the invention provides treatment methods combining the use of the standard chemotherapy treatment with the infusion of ex vivo propagated T cells to improve tumor-free survival. In one embodiment, the invention provides methods combining CHOP and infusion of ex vivo propagated T calls to improve the outcome of canines receiving CHOP for spontaneously-occurring B-lineage NHL. The present invention therefore provides methods for augmenting a patient's immune response by the addition of polyclonal ex vivo propagated T cells after lympho-depleting chemotherapy, which provides an improved tumor-free survival rate.

As demonstrated in the Examples below, T-cell infusion, or add-back T-cell therapy, after chemotherapy improves tumor-free survival with implications for NHL, as well as other tumor types. In particular, clinically-sufficient numbers of T cells were expanded from the peripheral blood (PB) of canines with B-lineage NHL on artificial antigen presenting cells in the presence of interleukin (IL)-2 and IL-21 to prepare an infusion product that was predominantly CD8⁺. The infused T cells were able to persist for at least 35 days, based on measurement of a fluorescent tag in PB, and traffic to sites of disease, resulting in improved tumor-free and overall survival. The present invention therefore provides an alternative approach for improving anti-tumor response and thereby tumor-free survival of patients with cancer, including NHL as well as other tumor types.

T-cell therapy in out-bred companion canines with cancer not only improves survival of the affected canines, but is useful as a model that informs on human immunotherapy of cancer, such as NHL.

Pharmaceutical Compositions

Ex vivo propagated T cells to be infused in accordance with the present invention can be incorporated into a pharmaceutical composition suitable for administration. Such compositions typically comprise the active compound and a pharmaceutically acceptable carrier suitable for administration of T cells. Such compositions and carriers are known in the art and would known to one of skill in the art.

A pharmaceutical composition of the invention is formulated to be compatible with its intended route of administration. “Administration” herein refers to the delivery of the active compound to a patient. Such a patient may be any mammal in need of treatment for lymphoma. In one embodiment, a patient in accordance with the present invention is a canine or human. Administration includes routes of administration which allow the compositions of the invention to perform their intended function. Any suitable administration method may be used in the present invention. Examples of routes of administration include subcutaneous, intravenous, intra-muscular, intra-arteriol, intra-peritoneal, intra-dermal, intra-tumoral or transdermal. Suitable modes of administration include use of intravenous infusion, solid implants, subcutaneous injection and oral administration in a form avoiding break down by stomach acids and digestive enzymes. In one embodiment, ex vivo propagated T cells may be provided by means of intravenous infusion.

In one embodiment, it may be advantageous to formulate compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

As used herein an “effective amount” or a “clinically-sufficient amount” includes those amounts of ex vivo propagated T cells which allow performance of the intended function, e.g., to increase anti-tumor response as compared to a patient not receiving T cells, as described herein. The effective amount may depend upon a number of factors, including biological activity, age, body weight, sex and general health. For example, 5×10⁷/m², 5×10⁸/m² or 3×10⁹ cells/m² ex vivo propagated T cells may be administered.

The timing of administration can also vary depending on a number of factors. For instance, timing can depend upon the health of the animal or stage of the disease. The timing of additional treatments may also affect the timing of the T-cell transfer therapy of the present invention. In particular, ex vivo propagated T cells can be administered to a patient during any time in which the patient is in need of treatment for lymphoma. T-cell administration can also accompany or be administered contemporaneously with other cancer treatments. Such other cancer treatments may comprise chemotherapy, immunotherapy, radiation, hematopoietic stem cell transplantation or combinations thereof. Ex vivo administered T cells may, in one embodiment, be administered prior to, concurrently (during) or after another cancer treatment.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Animals and Infusion Study Model

A. Eligibility for Clinical Trial

Client-owned canines treated at Texas A&M University (TAMU) College of Veterinary Medicine participated with owner's written consent. Normal control patients were not restricted based on breed or health status. A diagnosis of B-cell lineage NHL was the inclusion criterion for entering the trial, while the exclusion criterion for adoptive immunotherapy included: canines too ill to proceed in the opinion of the veterinary staff, known allergies to bovine or murine products, and insufficient T-cell ex vivo expansion. Ten canines were enrolled and eight infused. Before infusions, one patient died of disease not related to cancer or treatments, and another was lost during follow-up.

B. Control Canines

Healthy PB donors were chosen at random from canines seen at TAMU during 2010 for annual checkups. Canines with biopsy/cytology-proven B-lineage NHL, with stage IV to V disease, seen at TAMU between 2007 and 2010, and only treated with CHOP were used as controls for survival curves (n=12).

C. Study Design

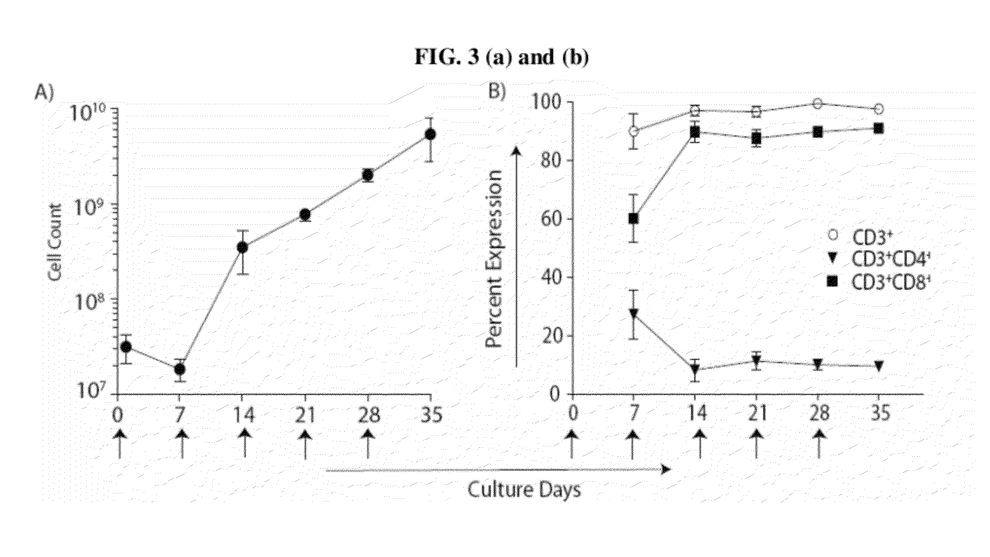

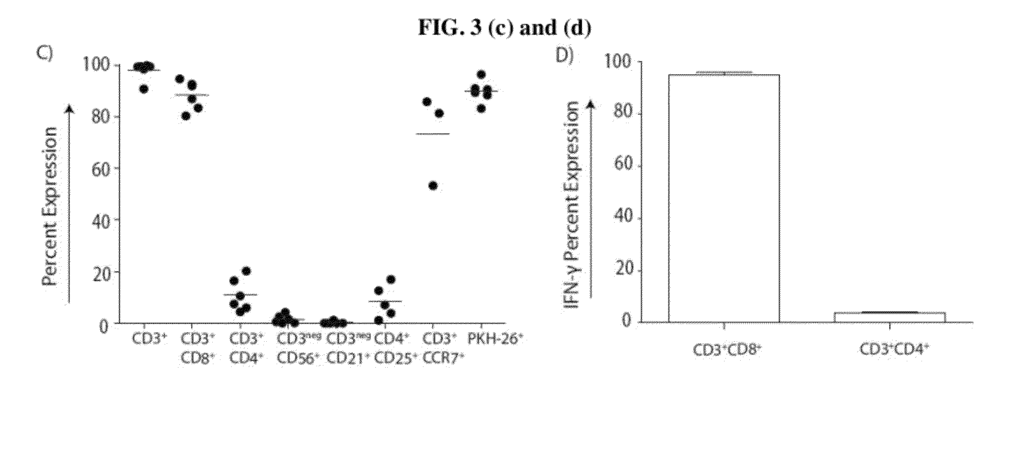

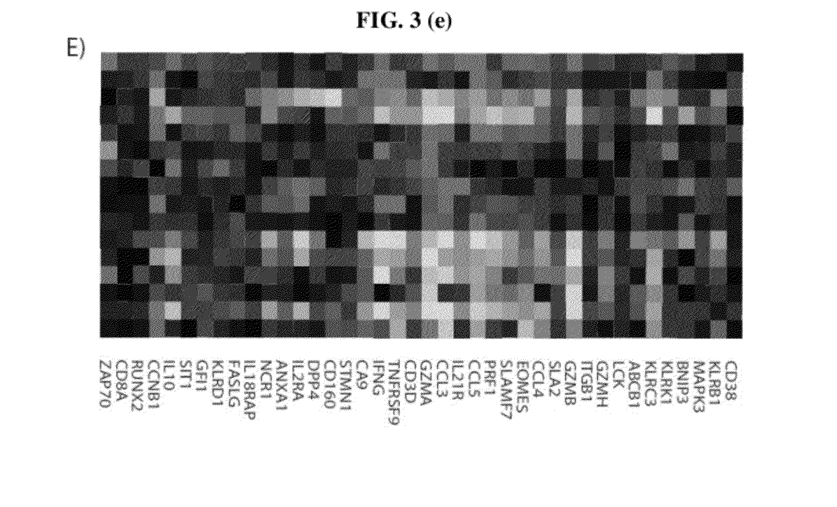

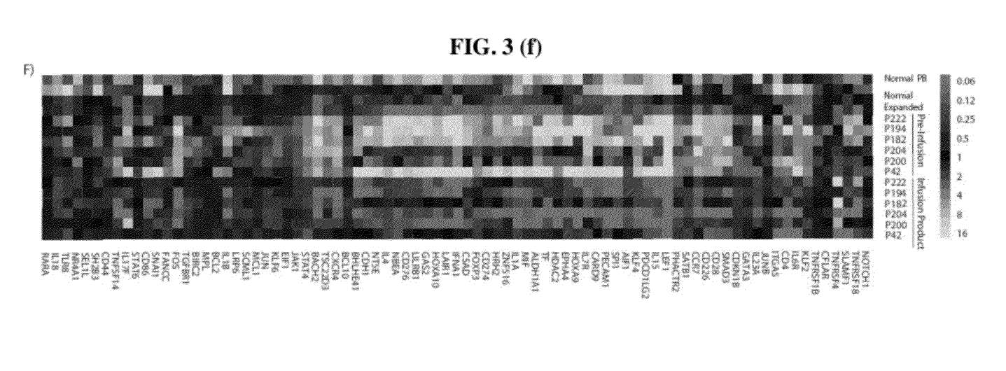

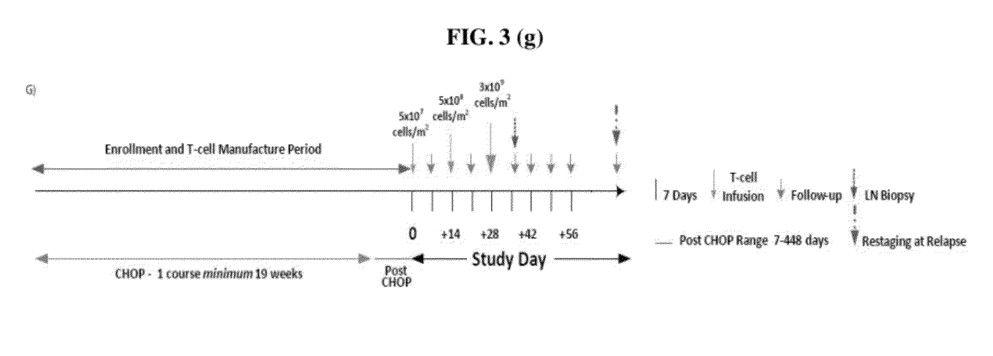

Upon enrollment, fine needle aspirates (FNA) of lymph nodes (LNs) verified B-lineage NHL. Trial participants are described in Table 1. All participants (and historical controls with NHL) received 19-weeks of CHOP prior to T-cell infusions (Table 2). Blood samples were taken at the time of enrollment, pre- and 3 hours post each infusion, as well as, throughout the trial (FIG. 3). Patients were enrolled regardless of treatment stage (before, during or after CHOP protocol). Using an intra-patient dose escalation scheme, three T-cell doses (up to 5×10⁷/m², 5×10⁸/m², and 3×10⁹ cells/m²) were infused based on canine body surface area (BSA) (FIG. 3). Study Day 0 was defined as the day T cells were infused. T cells were first infused at a median of 14 days (range 7 to 448 days), after completion of CHOP. Two patients, P194 and P182, received their T-cell infusions 105 and 448 days, respectively, after CHOP. Patient P42 relapsed prior to initiation of adoptive immunotherapy and received mechlorethamine, vincristine, procarbazine, and prednisone (MOPP), L-asparaginase, tocerinib, and prednisone, concurrent with the T cell infusions, and was excluded from reporting tumor-free survival and serum TK analysis. P42 achieved only stable disease with T-cell infusions, while all other participants were in remission after CHOP-based chemotherapy and at the time of infusion.

D. Statistical Methods

Both percentage and fold expansion means are shown as mean±standard error (SE). Further analysis used the Student's t-test with p-values less than 0.05 as significant. For the survival curves, Kaplan-Meier and Log Rank analyses were completed against historical controls with p-values less than 0.05 listed as statistically significant. GraphPad Prism version 5.0 for Windows was used for all statistical calculations (GraphPad Software, San Diego, Calif.).

TABLE 1
Patient characteristics that received T cells after CHOP

| | | | | | | | Staging at the | | No. days T cells |
|---|---|---|---|---|---|---|---|---|---|
| | | | Age | BSA | Initial | | time of 1^{st} T- | No. T-cell | cultured before |
| UPN | Breed | Sex | (yrs) | (m²) | Staging | Chemotherapy | cell infusion | Infusions | cryopreservation |
| P42 | Rottweiler | F | 5 | 1.2 | IV | CHOP, | PD | 3 | 35 |
| | | | | | | P, M, T | | | |
| P182 | Labrador | F | 6 | 1.1 | IV | CHOP | CR | 3 | 35 |
| | Retriever | | | | | | | | |
| P194 | Border | F | 6 | 0.9 | IV | CHOP | CR | 3 | 28 |
| | Collie | | | | | | | | |
| P200 | Rottweiler | M | 4 | 1.3 | V | CHOP | CR | 3 | 33 |
| P204 | Labrador | F | 11 | 0.9 | IV | CHOP | CR | 3 | 24 |
| | Retriever | | | | | | | | |
| P222 | Cocker | F | 5 | 0.5 | IV | CHOP | CR | 3 | 27 |
| | Spaniel | | | | | | | | |
| P249 | Labrador | F | 9 | 1.08 | IV | CHOP | CR | 2 | 35 |
| | Retriever | | | | | | | | |
| P263 | Sheltie | M | 6 | 0.66 | IV | CHOP | CR | 1 | 35 |

T = Toceranib; M = MOPP; P = Prednisone; CR = Complete Remission; PD = Progressive Disease
The average T-cell dose for the infusions administered to 8 dogs was 6.45 × 10⁸ ± 1.65 × 10⁸ cells/m² (mean ± s.e.m.) (range: 5 × 10⁷ to 2.5 × 10⁹ cells/m²).
Patient P42 relapsed prior to initiation of adoptive immunotherapy and received mechlorethamine, vincristine, procarbazine, and prednisone (MOPP), L-asparaginase, toceranib, and prednisone, concurrent with the T-cell infusions. This dog achieved stable disease after T-cell therapy.

TABLE 2
Summary of drugs used for chemotherapy for patients

| Drug | Vendor | Dosing (mL/m²) | |
|---|---|---|---|
| Cycolphosphamide | Amatheon (Miami, FL) | 240-250 | mg/m² |
| Vincristine | Cardinal Distribution (Dublin, | 0.5-0.7 | mg/m² |
| | OH) | | |
| Doxorubicin | Cardinal Distribution | 30 | mg/m² |
| Prednisone | Cardinal Distribution | 2 | mg/kg |
| Lomustine | Cardinal Distribution | 60-70 | mg/m² |
| L-asparaginase | Cardinal Distribution | 10,000 | IU/m² |
| Mechlorethamine | Amatheon | 3 | mg/m² |
| Tocerinib | Pfizer (New York, New York) | 3-3.25 | mg/kg |

### Example 2

### Ex Vivo T-Cell Propagation

A. Materials and Methods

1. Isolation of Mononuclear Cells and Serum from PB

Canine PB mononuclear cells (PBMC), diluted 1:10 with EDTA/PBS CliniMacs (Miltenyi Biotec, Auburn, Calif.) were isolated by density-gradient centrifugation over Ficoll-Paque-Plus (GE Healthcare BioSciences, Piscataway, N.J.) and cryopreserved in HyQ RPMI 1640 (HyClone, Logan Utah), 10% heat-inactivated Fetal Bovine Serum (FBS, HyClone), and 10% DMSO (Sigma, St. Louis, Mo.), termed freeze media. Serum was stored at −80° C.

2. Flow Cytometry:

Fluorochrome-conjugated canine-specific monoclonal antibodies (mAb) (AbD Serotec, Raleigh, N.C.) were used at a 1:25 dilution in 2% FBS and 0.1% sodium azide (Sigma) in PBS (Sigma), termed FACS buffer: mouse anti-dog CD3 (clone: CA17.2Al2, catalog number: MCA1774F), rat anti-dog CD4 (YKIX302.9, MCA1038A647), rat anti-dog CD8 (YCATE55.9, MCA1039PE), mouse anti-dog CD21 (AbD Serotec, CA2.1D6, MCA1781PE), rat anti-dog CD5 (YKIX322.3, MCA1037PE), mouse anti-human CD25 (Dako, Carpinteria, Calif., ACT-1, F0801), mouse anti-human CD56 (Dako, MOC-1, R7127), and mouse anti-dog CD21(CA2.1D6, MCA1781 PE). Other antibodies were used at 1:25 dilution: human anti-CCR7 (BD Pharmingen, FL 3D12, 552176) and human anti-CD32 (BD Pharmingen, 18.26, 559769). Cells were stained for 30 minutes with mAbs at 4° C. in FACS buffer. Granzyme B staining was undertaken on T cells fixed for 20 minutes in BD Cytofix/Cytoperm solution (BD Biosciences, San Jose, Calif.). After washing in BD perm wash buffer, cells were incubated with mouse anti-human granzyme B (BD Pharmingen, GB11, 560211) and isotype control (BD Pharmingen, mouse anti-Rat IgG2a, 558067) for 30 minutes. To measure IFN-γ expression, cells were incubated with golgi plug (BD Biosciences) in a 1:1000 dilution in media for 4 hours at 37° C. before washing and fixation as described above, and stained for 30 minutes with mouse anti-bovine IFN-γ mAb (AbD Serotec, CC302, MCA1783PE), at 4° C. in 1:10 dilution in BD perm wash buffer. Data was acquired with the FACS Calibur (BD Bioscience) and Cell Quest Version 2.0 (BD Biosciences). Data was analyzed with FCS Express Version 3.0 (De Novo Software, Thornhill, Ontario, Canada).

3. aAPCs

The human cell line K562 (homogenously expressing endogenous marker CD32) was transduced with lentivirus to co-express human CD19, CD64, CD86, CD137L, and membrane bound human IL-15 (co-expressed with EGFP), and cloned by limiting dilution. CLN4 was fingerprinted at MDACC using short tandem repeat PCR and proven to be derived from K562. CLN4 was γ-irradiated (100 Gy) prior to loading with 1 μL/10⁶ cells of mAb specific for human CD3 (OKT3 Orthoclone, Toronto, Ontario, Canada), cryopreserved in freeze media, and could be used immediately after thawing. Flow cytometry confirmed the loading of OKT3 using an anti-F(ab′)₂ specific antibody (1:100 dilution) (Jackson ImmunoResearch Laboratories, West Grove, Pa., R-Phycoerythrin-conjugated F(ab′)₂ fragment of Goat anti-Mouse IgG fragment F(ab′)₂ Specific, 115-116-072), and stable expression of introduced T-cell co-stimulatory molecules.

4. Numeric Expansion of T Cells on γ-Irradiated aAPC

PBMC and T cells were cultured in HyQ RPMI 1640 (HyClone) supplemented with 2 mmol/L Glutamax-1 (Life Technologies-Invitrogen, Carlsbad, Calif.) and 10% heat-inactivated defined FBS. T cells could be activated for sustained proliferation upon cross-linking CD3 by OKT3-loaded γ-irradiated CLN4. T cells, maintained at 7×10⁵ cells/mL, were cultured at a 2:1 (T cell:aAPC) ratio and re-stimulated every 7-10 days with aAPC. Recombinant human (rh) interleukin (IL)-21 (rhIL-21, eBiosciences, San Diego, Calif.) was added (30 ng/mL) three-times-per-week for the first seven days. rhIL-21 and rhIL-2 (Invitrogen, Carlsbad, Calif.) at 100 U/mL were added three-times-per-week, for subsequent stimulations with thawed aAPC. Viable T cells were enumerated every 7 days by Trypan blue exclusion using the Auto T4 Cell Counter Cellometer (Nexcelom Bioscience, Lawrence, Mass.) and cryopreserved at 4×10⁷ cells/mL in freezing media.

5. Fluorescent-Labeling of Propagated T Cells

T cells were labeled with the red fluorescent dye, PKH-26, using the Cell Linker Kit for General Cell Membrane Labeling (Sigma, PKH26GL) according to manufacture's instructions at room temperature. Briefly, T cells were re-suspended in Diluent C at 3×10⁷ cells/mL, before adding another Diluent C solution containing a mixture of equal number of mLs as the cell suspension and PKH-26 at a dilution of 4 μL/mL. The two solutions were mixed together and incubated for 5 minutes, before adding an equal volume of FBS to stop the reaction. Cells were washed and centrifuged three times at 200×g for 10 minutes.

B. Results

1. PB T-Cell Immunophenotype Obtained from Healthy and NHL Diagnosed Canines

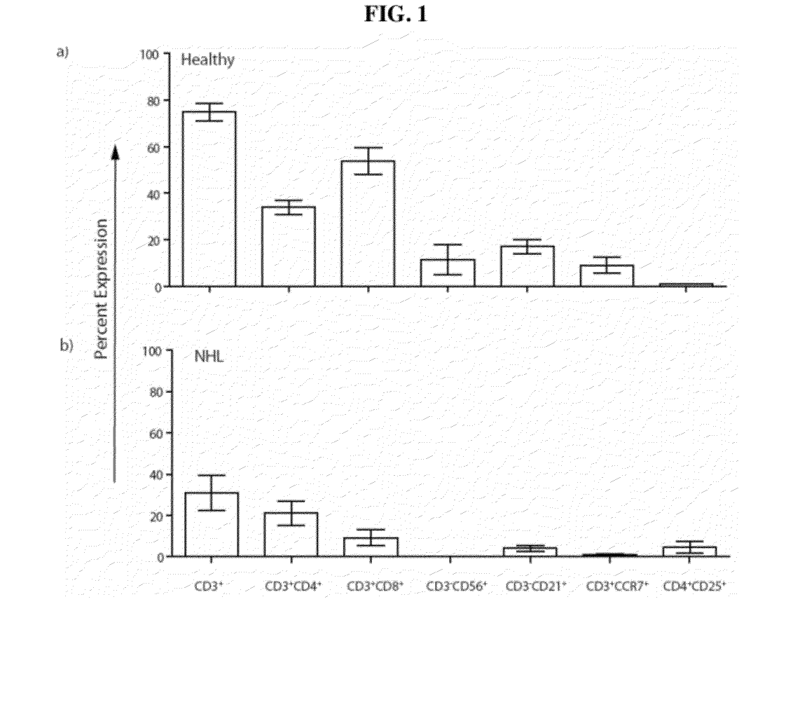

The immunophenotype of T cells in PB was compared between healthy canines and canines with NHL before their treatment with CHOP (FIG. 1). In healthy canines the average CD3⁺ population was 74±4% (mean±SE). CD3⁺CD4⁺ T cells (33±3%) encompassed a reduced percentage of the T-cell population compared to CD3⁺CD8⁺ T cells (54±6%). Among the CD3⁺CD4⁺ population, the mean CD25⁺ percentage was 1.2±0.03%, which is consistent with low circulating numbers of regulatory T cells. Natural killer cells (NK), described as CD3^{neg}CD56⁺, comprised an average of 11±6%, while CD3^{neg}CD21⁺ B cells were present at 17±3%. In comparison, there was a decrease in the percentage of CD3⁺ T cells (30±8%) in PBMC from canines with NHL (FIG. 1), which was statistically different than the percentage of CD3⁺ T cells in healthy canines (p=0.003). PBMC from canines with NHL had an increased mean percentage of CD3⁺CD4⁺ T cells (21±6%) compared to normal donors and a decreased percentage CD3⁺CD8⁺ T-cell population (9±4%). The difference in CD3⁺CD8⁺ T cells between healthy and canines with NHL was statistically significant (p=0.0006). CCR7 is a marker for T-cell LN migration and memory phenotype and the mean CD3⁺ CCR7⁺ T cells in PBMC from healthy canines was 9±3% and 0.8±0.6% in PBMC from canines with NHL (FIG. 1). The NK populations (0.2±0.05%) were also decreased and B cell population decreased (4.1±1.3%), p=0.13 and p=0.008, respectively. A slight increase in the CD4⁺CD25⁺ population, which presumably includes regulatory T cells, was observed in PBMC from canines with NHL (4±3%, p=0.18). Overall, lower numbers T and NK cells in canines with NHL suggested a state of immune suppression.

2. Canine T Cells can be Propagated on aAPC when Co-Cultured with rhIL-21

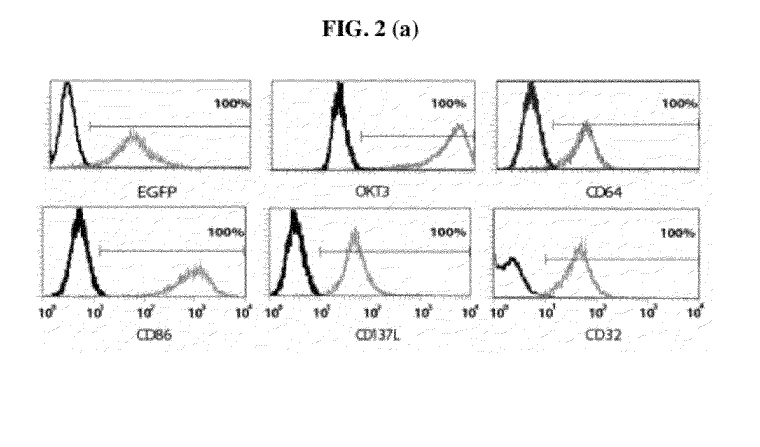

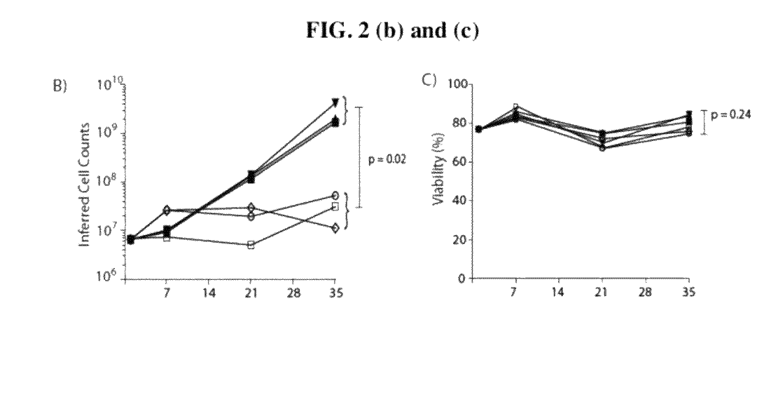

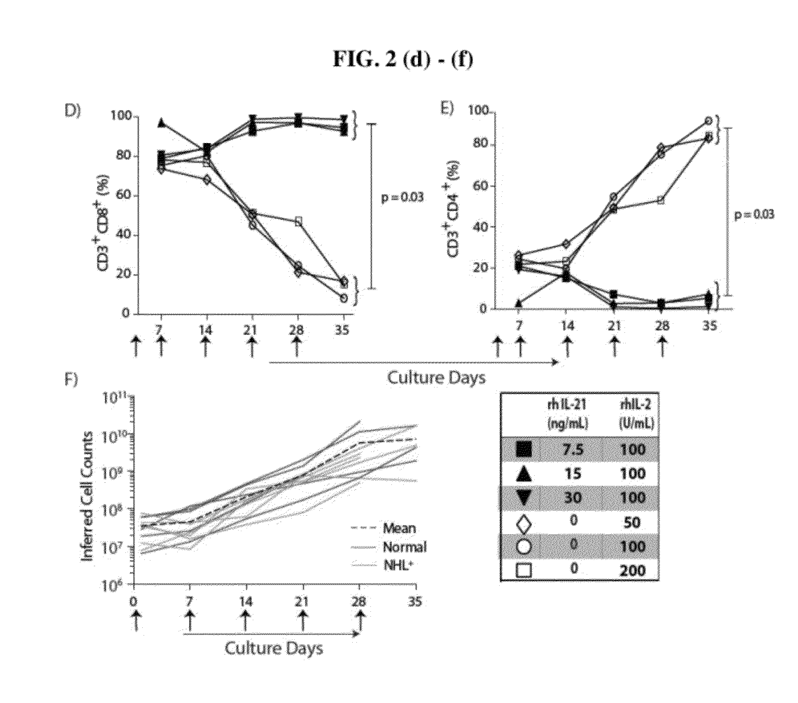

Canine T cells from healthy donors were numerically expanded from PBMC on γ-irradiated aAPC CLN4 loaded via CD64 with OKT3 (FIG. 2). PB-derived T cells from healthy canines propagated in the presence of graded doses of rhIL-2 had limited success, for while the cultured T cells remained viable, they demonstrated only a 6.5±2.4 fold expansion over 35 days (n=2). Therefore, it was determined whether additional signaling through the common γ-receptor could sustain canine T-cell proliferation. The addition of graded doses of rhIL-21, in addition to rhIL-2, resulted in 399±124 fold expansion over 35 days (n=2) (313±75 fold expansion after 28 days) which was significantly greater than the expansion achieved with only rhIL-2 (FIG. 2). The mean fold expansion correlated with an increased rhIL-21 concentration (FIG. 2). There were no statistically-significant differences in viability between the cultures containing rhIL-2 alone and rhIL-2 and rhIL-21 (p=0.24) (FIG. 2). These data demonstrate that a combination of rhIL-21 and rhIL-2, in coordination with cross-linking CD3 to OKT3 are required to sustain proliferation of canine T cells on CLN4 aAPC.

3. rhIL-21 Selectively Propagates CD8⁺ Canine T Cells

The addition of rhIL-21 selectively propagates human CD8⁺ T cells which is desirable as the cytolytic effector function of infused CD8⁺ T cells can control tumor growth. The influence of this cytokine in combination with rhIL-2 on the selective outgrowth of CD8⁺ versus CD4⁺ T cells was investigated. While there was a decrease in the average expression of CD3⁺CD8⁺ T cells from 76±1% on day 7 to 14±3% on day 35 in T-cell cultures with only rhIL-2, the average expression of CD3⁺CD8⁺ T cells cultured with both rhIL-2 and rhIL-21 increased from 73±2% on day 7 to 95±2% on day 35 (FIG. 2). These data are consistent with the ability of rhIL-21 to preferentially support outgrowth of CD8⁺ T cells which has practical implications for following the fate of infused T cells as canines treated with CHOP preferentially recover CD4⁺ T cells.

4. Propagation of T Cells from Canines with B-Lineage NHL

Late stage presentation of NHL and treatment with CHOP led to T-cell lymphopenia characterized by decreased numbers of CD3⁺ T cells and low numbers of CD8⁺ T cells, similar to observations in humans after CHOP. The culture conditions established to selectively expand CD8⁺ T cells from healthy canines were applied to propagate T cells from canines with NHL. After 28 days of co-culture on CLN4 and rhIL-21/2, T cells underwent an average 124±44 fold expansion (n=6), which was less than achieved for T cells from healthy canines (221±39 fold expansion, p=0.04, n=6) (FIG. 2). These data demonstrate that the platform technology of every-7 day addition of aAPC with rhIL-21 and rhIL-2 can sustain the proliferation of CD8⁺ T cells. Reduced ex vivo expansion suggested possible T-cell dysfunction. These experiments showed that was possible to drive effector T-cell expansion preferentially from lymphoma donors and provided the foundation for ex vivo expanded T-cell infusions to treat lymphoma diagnosed canines.

5. Propagation of T Cells for Infusion

PB T cells were recursively stimulated on CLN4 in the presence of rhIL-2 and rhIL-21 for up to 35 days. During this time, T cells underwent a mean 109±15 fold expansion (FIG. 3). Seven days post initial stimulation cultures were 89±6% CD3⁺ with 60±8% CD3⁺CD8⁺, while CD3⁺CD4⁺ T cells decreased to 27±8%. At day 14 and beyond, cultures were predominantly CD3⁺CD8⁺ (90±4%) and mean CD3⁺CD4⁺ population decreased to 8±4% (FIG. 3). The infusion product analyzed for 6 of the 8 treated dogs at the time of cryopreservation contained an average 98±1% CD3⁺ T cells; 88±2% CD3⁺CD8⁺; 11±3% CD3⁺CD4⁺; 93±0.7% CD3⁺CD5⁺; 0.2±0.2% CD3⁻CD21⁺; 8±3% CD4⁺CD25⁺; 73±10% CD3⁺CCR7⁺; and 90±2% PKH-26⁺ (FIG. 3). Three patients with NHL, not yet infused, were analyzed for endogenous IFN-γ expression after OKT3-driven expansion. The majority of CD8⁺ and CD4⁺ T cells expressed IFN-γ, 99.9±1% and 3.7±0.6%, respectively (FIG. 3). It was noted that of the propagated CD4⁺ T cells, 8±3% co-expressed CD25 and their reduced production of IFN-γ is consistent with a regulatory function. CLN4 can support rapid expansion and activation through IFN-γ expression of sufficient CD3⁺CD8⁺ numbers from NHL-positive patients. The decreased PB CD8⁺ T-cell population was exploited in our trial through preferentially infusing CD8⁺ T cells.

### Example 3

### Patient T-Cell Treatment

A. Materials and Methods

1. Infusion of Propagated T Cells

Cyropreserved products were shipped on dry ice to TAMU for storage and infusion. T cells were thawed at 37° C. and intravenously infused without washing within 10 minutes at a rate not exceeding 2 mL per 3 minutes. Adverse reactions were determined by the Veterinary Co-Operative Oncology Group (VCOG) scale.

2. Complete Blood Counts (CBC)

Canine CBC were analyzed at TAMU using Abbott Cell Dyn 3700 (Abbott Park, Ill.) to obtain absolute neutrophil counts (ANC), absolute lymphocyte counts (ALC), and platelet counts.

3. Immunohistochemistry

Popliteal LNs were biopsied and sections snap-frozen and/or formalin-fixed. Frozen sections were evaluated for the presence of CD3 and CD79a using an alkaline phosphatase detection system using an autostainer (Dako), which dispensed 300 μL of reagent per slide. Slides were either acetone or formalin-fixed for 5 minutes prior to staining. Universal Block (KPL Labs, Gaithersburg, Md.) was applied to block endogenous phosphatase. Slides were incubated for 60 minutes with the primary antibodies diluted 1:200 in Da Vinci Green Diluent (Biocare Medical, Concord, Calif.): rabbit anti-human CD3 (Dako, A0452) and mouse anti-dog CD79a (Dako, M7051, HM57). Secondary antibodies, biotinylated goat anti-mouse (KPL Labs, 71-00-29) or goat anti-rabbit (KPL Labs, 71-00-30), were incubated for 60 minutes before streptavidin phosphatase (KPL Labs, 71-00-45). Vulcan fast red (Biocare Medical) was used to detect the antibody binding sites. Tissues were counterstained with hematoxylin (Biocare Medical).

B. Results

1. Infusion and Persistence of Infused T Cells

The majority of the seven recorded adverse events were Grade I or 2 and all adverse events were observed within a 72-hour period after the second infusion (Table 3). One canine with a Grade III gastrointestinal adverse event (within 72 hours of infusion) required hospitalization for dehydration (24 hours).

TABLE 3
Number of Canines with Adverse Events
Possibly Attributed to T-cell Infusion

| | Dose 1 | | Dose 2 | | Dose 3 | |
|---|---|---|---|---|---|---|
| | Grade | Grade | Grade | Grade | Grade | Grade |
| Symptom | I, II | III, IV | I, II | III, IV | I, II | III, IV |
| Lethargy | 0 | 0 | 0 | 0 | 0 | 0 |
| Nausea | 0 | 1 | 0 | 0 | 0 | 0 |
| Diarrhea | 0 | 0 | 2 | 1 | 0 | 0 |
| Vomiting | 0 | 0 | 2 | 1 | 0 | 0 |

Grading based on Veterinary Co-operative Oncology Group Scale

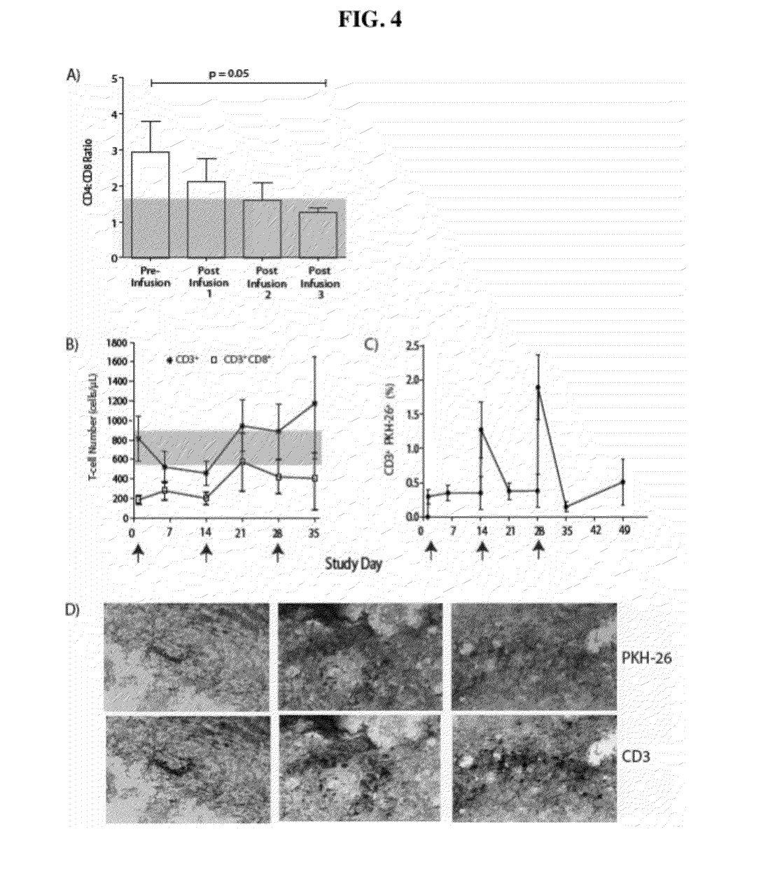

To evaluate the persistence of infused T cells, the PBMC CD4⁺ and CD8⁺ T cell percentage prior to adoptive immunotherapy was determined. The majority of enrolled patients with NHL had a dominant CD3⁺CD4⁺ T-cell phenotype before and after CHOP (FIG. 1). Since lymphopenia may be conducive to the homeostatic expansion of infused T cells, the content of PBMC CD3⁺ T cells was determined and it was demonstrated that there was a lower than normal (FIG. 1) mean percentage of CD3⁺ T cells in PBMCs from canines with NHL. By infusing a product which was primarily CD8⁺ cells, the CD4:CD8 ratio in PBMC could be serially assessed as a measure of infused T-cell persistence (FIG. 4 and Table 4). The mean normal CD4:CD8 ratio is 1.6:1±0.2 (n=4), while the mean pre-infusion ratio was 3:1. After three subsequent infusions, the mean ratio decreased to 1:1.2, a significant change (p=0.05) in the mean ratio to infusions (FIG. 4). These ratios remained stable after infusion. P42 had increased CD3⁺CD4⁺ percentages and CD4:CD8 ratios that decreased after subsequent infusions and allowed stable disease development. This patient was unlike the other participants, who were in remission before infusion. Because of her advanced disease, the significance is not as great, but regardless was still included in the statistics. A trend was observed in P200 and P222 during relapse prior to chemotherapy rescue as the CD4:CD8 ratio and the CD3⁺CD4⁺ percentage of the ALC increased.

TABLE 4
CBC comparisons between healthy controls and infusion patients

| | Average WBC(10³/μL) | Average ALC(cells/μL) | Average ANC (cells/μL) | Average Platelet Count (cells/μL) | (n) |
|---|---|---|---|---|---|
| Healthy Control | 9.21 ± 0.8 | 2,800 ± 547 | 5,912 ± 385 | 293,333 ± 37,243 | 6 |
| Enrollment/Pre-CHOP | 8.7 ± 1 | 1,450 ± 264 | 6,304 ± 701 | 276,750 ± 69,556 | 5 |
| Study Day-14 | 4 ± 1 | 890 ± 368 | 3,630 ± 753 | 363,666 ± 162,120 | 3 |
| Study Day 0 | 7.3 ± 1 | 1,256 ± 324 | 5,491 ± 900 | 228,200 ± 56,045 | 6 |
| Study Day 35 | 11.3 ± 3 | 1,479 ± 539 | 10,064 ± 4,096 | 179,000 ± 37,447 | 6 |

As a biological marker for the persistence of infused T cells, the CD8⁺ ALC was measured as compared to CD4⁺ ALC. The CD3⁺CD8⁺ population increase coincided with each subsequent, escalating infusion dose (FIG. 4). The average CD3⁺CD4⁺ and CD3⁺CD8⁺ pre-infusion ALC were 585±223 cells/μL and 188±44 cells/μL, respectively. By study day 35, the average CD3⁺CD4⁺ and CD3⁺CD8⁺ ALC were 305±166 cells/μL and 469±142 cells/μL, respectively. The decrease in CD3⁺CD4⁺ T cells after the first infusion was only temporary, as this T-cell population began to increase to normal homeostatic levels. However, the CD3⁺CD8⁺ ALC remained elevated in those patients in complete remission. The ALC CD3⁺ T-cell percentage increased above pre-infusion levels during the same time frame, from 64% to 79% (FIG. 4). The increased T-cell numbers in PB can result only from the infusions. The ALC modulation may represent homeostatic adjustment to incorporate the influx of activated T cells and may support faster recovery from lymphodepleting chemotherapy.

To directly examine the persistence of infused T cells, the infusion products were labeled with PKH-26. PKH-26⁺CD3⁺ T cells were detected in the PB at 3 hours, and study days 7, 14, and 49 (FIG. 4). The increased total CD3⁺ T-cell ALC correlated with the detected increase in PKH-26 labeled CD3⁺ T cells in PB (FIG. 4). The average percentage of PKH-26⁺ T cells detected in the PB 3 hours post infusions 1, 2, and 3 were 0.3±0.1%, 1.3±0.4%, and 1.9±0.4%, respectively. The percentages and mean fluorescent intensity decreased 7 days after each infusion, but they were still at detectable levels up to 21 days after the last infusion. The decreased levels between 7 and 14 days post infusion may be attributed to infusion product expansion, as the PKH-26 signal is reduced during subsequent cell divisions, and/or trafficking to the tumor or tissues.

2. Infused T Cells Home to Tumor

LN biopsies were sampled 10 days after the last infusion to determine if infused T cells trafficked to the tumor sites. Immunophenotype analysis demonstrated that the majority of cells in the LN were CD79a⁺, which is a marker for canine B cells. Using fluorescent microscopy, PKH-26 stained T cells were located throughout the LN and these sections were co-stained with anti-canine CD3 (FIG. 4). The CD3⁺CD8⁺ infused product trafficked to the LN and persisted at the tumor site, as observed by the dual staining.

3. T-Cell Infusions after CHOP Improve Tumor-Free Survival

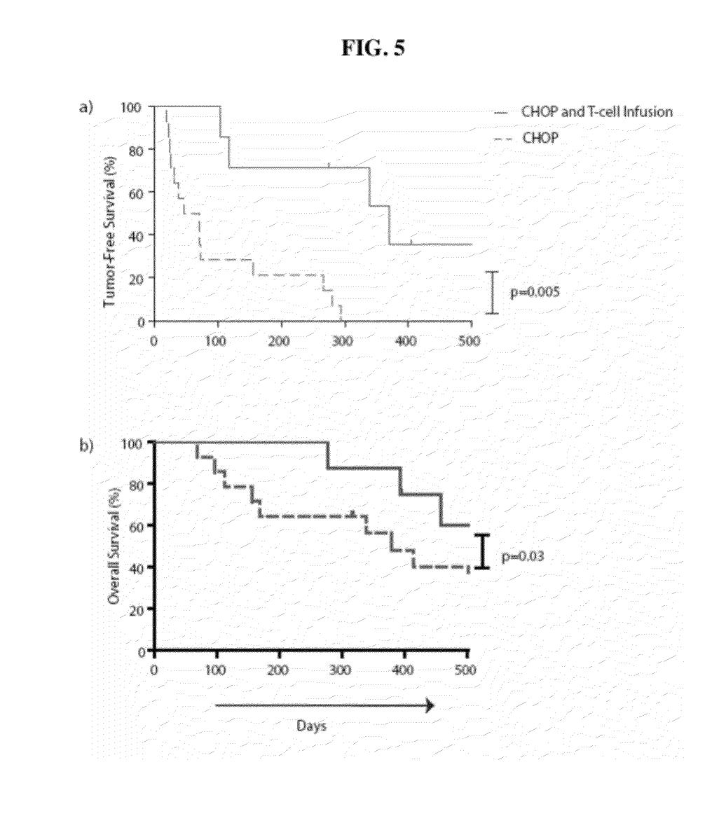

To evaluate the ability of ex vivo-propagated T cells to impact canine survival, the 8 dogs that received CHOP and autologous T cells were compared with a cohort of stage-matched canines with NHL that received only CHOP. Both cohorts were followed for 500 days post initial diagnosis of NHL or obtaining CR for the analysis of tumor-free survival. As expected, 7 of 8 dogs that received T-cell infusions achieved a prior complete remission (CR) from CHOP. However, it was observed that the infusion of T cells after CHOP resulted in marked improvements in overall and tumor-free survival and that this was evident when only 8 dogs had been infused (FIG. 5). The historical controls which were matched for age, size, disease stage, and achieving CR after CHOP with the canines that received T cells after CHOP. This control population had a median overall survival (n=12) of 167 days (range from 68 to 413 days) upon initial diagnosis of NHL and a median tumor-free survival (n=12) of 71 days (range from 23 to 293 days) following the CR achieved after receiving CHOP. Significantly, for the 8 canines receiving CHOP combined with T-cell infusions the median overall survival was improved to 392 days (range 277 to 458 days) and the tumor-free survival improved to 338 days (range 104 to 369 days). The hazard ratio and p-value for the log rank test of overall survival were 4.3 and 0.03, respectively. T-cell infusions significantly increased the period of tumor-free survival when used in conjunction with CHOP therapy (p=0.005) which implies a clinical benefit to dogs with NHL that received immunotherapy.

### Example 4

### Gene Expression Assay

A custom panel was generated (Table 5) to quantify 346 canine T-cell specific genes with a single non-enzymatic reaction using the nCounter Prep Station and Digital Analyzer (NanoString Technologies, Model NCT-SYST-120, Seattle, Wash.). PB T cells were isolated flow cytometrically using mouse anti-dog CD3 (AbD Serotec) and a BD FACS Aria II high speed sorter (BD Biosciences) prior to analysis. Propagated T cells (99% CD3⁺ and less than 1% CD32⁺) were analyzed after 28 days of culture and 7 days from the last addition of aAPC. Propagated T cells were cryopreserved, thawed at 37° C., and rested with cytokines and media for 2 hours before flash freezing in liquid nitrogen. Sample mRNA was isolated using the AllPrep DNA/RNA Mini kit according to manufacture's instructions (Qiagen, Valencia, Calif.) before gene profiling. Briefly, two sequence-specific gene probes, one mRNA target sequence-specific capture probe, and a second mRNA target sequence-specific fluorescent-labeled color-coded probe were produced for each of the selected genes. 30,000 cell equivalents of each sample were assayed as previously described in extensive detail. Probe hybridization was also performed as previously described in extensive detail.

For samples meeting a performance criterion based on positive control probes, probe counts for genes of interest (experimental probes) and “housekeeping” genes (normalization probes) were corrected by subtracting the integer-approximated mean of negative control probe counts in each sample, setting values of 0 or less to 1. Differential probe expression between pairs of samples was determined using a statistical test developed for “digital” gene expression profiling. In brief, from two libraries 1 and 2 from which N1 and N2 total numbers of clones have been randomly sampled, the probability of finding x and y numbers of clones for a particular cDNA in libraries 1 and 2 respectively, under the null hypothesis of identical expression of that cDNA, is given by the formula:

$$p ( y \vert x ) = {( \frac{{N}_{2}}{{N}_{1}} )}^{y} \frac{( x + y ) !}{x ! y ! {( 1 + \frac{{N}_{2}}{{N}_{1}} )}^{( x + y + 1 )}}$$

One cannot simply use the total number of experimental probe counts in two samples as estimates of N1 and N2, because experimental probes in most applications of the nCounter (as in ours) are chosen with a particular biology in mind, e.g., T-cell immune function, and are not necessarily equivalent in two samples. Therefore, N1 and N2 were estimated as the geometric mean of corrected values for those normalization probes clearly above background, and x and y are corrected experimental probe counts. Gene expression values for experimental probes were estimated by normalization of corrected probe counts, multiplying these by the ratio of the maximum normalization probe mean to each sample's normalization probe mean. Significant differential expression was defined by a combination of p<0.001 in the formula above and a fold-change ≧2. Heat-mapping of normalized values for differentially-expressed experimental probes used hierarchical clustering and TreeView software version 1.1.

The infusion product and pre-expansion PB T cells from each patient were compared to determine if aAPC stimulation conferred a cytolytic genotype and if the genotypic changes were similar to those observed in normal donors. Genes, undergoing a significant change (p<0.001, fold-change >2), were identified for each normal donor and patient with NHL and then filtered for uniformity across samples. Forty genes were up-regulated in at least 5/6 NHL patients and in both normal donor T cells after non-specific expansion (FIG. 3). Genes associated with T-cell activation, proliferation, signaling, as well as, cytoxicity were primarily up-regulated. Among these genes were granzyme B, H, A, IFN-γ, and perforin. Dosing with exogenous rhIL-21 and rhIL-2 influenced the increased expression of both IL-2Ra and IL-21 receptor. EOMES, DPP4, TNFRSF9, LCK and ZAP70 were also increased after OKT3-CD3 cross linking during expansion supporting increased T cell activation and proliferation. Chemokines associated with T-cell trafficking toward sites of inflammation and the LNs (CCL3, CCL4 and CCL5) were up-regulated in all samples after four stimulations. FasL was also expressed in the infusion product. Both normal donors and patients with NHL expansion products were similar in terms of cytotoxic gene expression.

Similarly, a set of 83 mRNA species were down-regulated after expansion in at least 5/6 canines with NHL and healthy subjects. Genes associated with adhesion, trafficking, and homing (ITGA5, CD226, CCR7, PECAM-1, CXCR4, CD44, and SEL1L) were down-regulated in the infusion product. Due to the incomplete nature of ex vivo stimulation, gene expression of several cytokines secreted by other cell types, but targeted to T cells, (IL-1, IL-6R, IL-15, IL-7R, IL-4, IL-17F, and IL-18) were decreased. Signaling proteins regulating the expression of these cytokines (JAK1, STAT4, JUN, JUN B, and SMAD3) were also down-regulated. FOXP3 was also decreased in the infusion product, which corresponded to the decreased number of CD4⁺ T cells expanded. A preferred infusion product cytolytic genotype was expanded after 28 days and was significantly similar to normal donor expanded T cells.

TABLE 5
Gene List for Canine T-cell Panel

| | | | | | | | |
|---|---|---|---|---|---|---|---|
| ABCB1 | ATP binding | EIF1 | Eukaryotic translation | KLRC2 | Killer cell lectin | STMN1 | Stathmin |
| | cassette | | initiation factor | | like receptor | | |
| ABCG2 | Mitoxantrone | ELF4 | Myeloid Elf-1 like | KLRC3 | Killer cell lectin | TBX21 | T-cell specific |
| | resistance | | factor | | like receptor | | T box |
| | protein | | | | subfamily C | | transcription |
| | | | | | | | factor |
| ADAM19 | A disintegrin | ENTPD1 | Ectonucleoside | KLRD1 | Killer cell lectin | TBXA2R | Thromboxane |
| | and | | triphosphate | | like receptor | | A2 receptor |
| | metalloprotease | | diphosphohydrolase | | subfamily | | |
| | domain | | | | | | |
| AGER | Advanced | EOMES | Eomesodermin | KLRF1 | Killer cell lectin | TCF7 | Transcription |
| | glycosylation | | | | like receptor | | factor 7, T-cell |
| | end product | | | | subfamily F | | specific |
| | specific | | | | | | |
| | regulator | | | | | | |
| AHNAK | Neuroblast | EPHA4 | Tyrosine protein | KLRG1 | Killer cell lectin | TDGF1 | Teratocarcinoma |
| | differentiation | | kinase receptor | | like receptor | | derived |
| | associated | | | | | | growth factor |
| | protein | | | | | | |
| AIF1 | Allograft | ETV6 | ets variant gene 6 | KLRK1 | Killer cell lectin | TDO2 | Tryptophanase |
| | inflammatory | | | | like receptor | | |
| | factor | | | | | | |
| AIMP2 | Aminoacyl | FADD | Mediator of receptor | LAIR1 | Leukocyte | TEK | Tyrosine |
| | tRNA | | induced toxicity | | associated | | kinase, |
| | synthetase | | | | immunoglobulin | | endothelial |
| | complex- | | | | like receptor 1 | | |
| | interacting | | | | | | |
| | multifunctional | | | | | | |
| | protein 2 | | | | | | |
| AKT1 | Protein kinase | FAM129A | Cell growth inhibiting | LCK | Lymphocyte | TERT | Telomerase |
| | B | | gene | | specific protein | | reverse |
| | | | | | tyrosine kinase | | transcriptase |
| ALDH1A1 | Aldehyde | FAM164A | Family with sequence | LDHA | Lactate | TF | Transferrin |
| | dehydrogenase | | similarity 164 member A | | dehydrogenase | | |
| | 1 family, | | | | | | |
| | member A1 | | | | | | |
| ANXA1 | Annexin 1 | FANCC | Fanconi anemia, | LEF1 | Lymphoid | TFRC | Transferrin |
| | | | complementation | | enhancer- | | receptor |
| | | | group | | binding factor 1 | | |
| APAF1 | Apoptotic | FAS | Tumor necrosis factor | LGALS3 | Lectin, | TGFA | Transforming |
| | peptidase | | receptor superfamily | | galactoside | | growth factor |
| | activating | | member | | binding, soluble | | |
| | factor | | | | | | |
| ARG1 | Arginase | FASLG | FAS ligand | LILRB1 | Leukocyte | TGFB1 | Transforming |
| | | | | | immunoglobulin | | growth factor |
| | | | | | like receptor | | |
| ARRB2 | Arrestin, beta | FLT1 | Vascular endothelial | LRP5 | Low density | TGFB2 | Transforming |
| | | | growth factor | | lipoprotein | | growth factor |
| | | | | | receptor-related | | |
| | | | | | protein 5 | | |
| ATM | Ataxia | FLT3LG | fms related tyrosine | LRP6 | Low density | TGFBR1 | Transforming |
| | telangiectasia | | kinase ligand 3 | | lipoprotein | | growth factor |
| | mutated | | | | receptor-related | | |
| | | | | | protein 6 | | |
| ATP2B4 | ATPase Ca++ | FOS | FBJ murine | LRRC32 | Leucine rich | TIE1 | Tyrosine |
| | transporting | | osteosarcoma viral | | repeat | | kinase with |
| | plasma | | oncogene homolog | | containing 32 | | immunoglobulin |
| | membrane 4 | | | | | | like and |
| | | | | | | | EGF like |
| | | | | | | | domains |
| B2M | Beta 2 | FOXP3 | Forkhead box P3 | MAD1L1 | Mitotic arrest | TLR2 | Toll like |
| | microglobulin | | | | deficient like 1 | | receptor 2 |
| B3GAT1 | CD57 | FYN | Proto oncogene | MAP2K1 | Mitogen | TLR8 | Toll like |
| | | | tyrosine protein kinase | | activated protein | | receptor 8 |
| | | | | | kinase kinase | | |
| BACH2 | Basic leucine | FZD1 | Frizzled homolog 1 | MAPK14 | Mitogen | TNF | Tumor |
| | zipper | | | | activated protein | | necrosis factor |
| | transcription | | | | kinase | | |
| | factor 2 | | | | | | |
| BAD | BCL2 | GAL3ST4 | Galactose | MAPK3 | Mitogen | TNFRSF18 | Tumor |
| | associated | | sulfotransferase | | activated protein | | necrosis factor |
| | agonist of cell | | | | kinase 3 | | receptor |
| | death | | | | | | superfamily, |
| | | | | | | | member 18 |
| BAG1 | BCL2- | GAS2 | Growth arrest specific | MAPK8 | Mitogen | TNFRSF1B | Tumor |
| | associated | | protein 2 | | activated protein | | necrosis factor |
| | athanogene | | | | kinase | | receptor |
| | | | | | | | superfamily |
| BATF | Basic leucine | GATA2 | Endothelial | MCL1 | Myeloid cell | TNFRSF4 | Tumor |
| | transcription | | transcription factor | | leukemia | | necrosis factor |
| | factor | | | | sequence | | receptor |
| | | | | | | | superfamily, |
| | | | | | | | member 4 |
| BAX | BCL2- | GATA3 | Trans acting T-cell | MIF | Macrophage | TNFRSF9 | Tumor |
| | associated X | | specific transcription | | migration | | necrosis factor |
| | protein | | factor | | inhibitory factor | | receptor |
| | | | | | | | superfamily |
| BCL10 | B cell | GFI1 | Growth factor | MMP14 | Matrix | TNFSF10 | Tumor |
| | CLL/lymphoma | | independent | | metallopeptidase | | necrosis factor |
| | | | | | 14 | | superfamily, |
| | | | | | | | member 10 |
| BCL2 | B-cell CLL | GLIPR1 | Glioma pathogenesis | MPL | Myeloproliferative | TNFSF14 | Tumor |
| | lymphoma 2 | | related protein | | leukemia virus | | Necrosis |
| | | | | | oncogene | | factor |
| | | | | | | | superfamily, |
| | | | | | | | member 14 |
| BCL2L1 | BCL2 like | GLO1 | Glyoxylase | MYB | Myeloblastosis | TOX | Thymocyte |
| | protein 1 | | | | viral oncogene | | selection |
| | | | | | homolog | | associated |
| | | | | | | | high mobility |
| | | | | | | | group box |
| BCL6 | Lymphoma | GSK3B | Glycogen synthase | MYC | Myelocytomatosis | TP53 | p53 tumor |
| | associated | | kinase | | viral oncogene | | suppressor |
| | zinc finger | | | | homolog | | |
| | gene on | | | | | | |
| | chromosome 3 | | | | | | |
| BHLHE41 | Basic helix | GZMB | Granzyme B | MYO6 | Myosin | TRAF1 | Tumor |
| | loop helix | | | | | | necrosis |
| | family, | | | | | | factor, type 1 |
| | member 41 | | | | | | |
| BID | Apoptotic | GZMH | Granzyme H | NBEA | Neurobeachin, | TRAF2 | Tumor |
| | death agonist | | | | lysosomal | | necrosis factor |
| | | | | | trafficking | | type 2 |
| | | | | | regulator | | |
| BIRC2 | Apoptosis | HCST | Hematopoietic cell | NCAM1 | Neural cell | TRAF3 | TNF receptor |
| | inhibitor | | signal transducer | | adhesion | | associated |
| | | | | | molecule | | factor |
| BMI1 | B lymphoma | HDAC1 | Histone deacetylase 1 | NCL | Nucleolin | TSC22D3 | TSC22 |
| | insertion | | | | | | domain family, |
| | region | | | | | | member 3 |
| BNIP3 | BCL2/adenovirus | HDAC2 | Histone deacetylase 2 | NCR1 | Natural | TSLP | Thymic |
| | E1B | | | | cytotoxicity | | stromal |
| | | | | | triggering | | lymphopoietin |
| | | | | | receptor | | |
| C11orf17 | Chromosome | HLA-A | MHC class I, antigen A | NCR2 | Natural | TYK2 | Tyrosine |
| | 11 open | | | | cytotoxicity | | kinase 2 |
| | reading frame | | | | triggering | | |
| | | | | | receptor | | |
| CA9 | Carbonic | HOXA10 | Homeobox protein A10 | NCR3 | Natural | TYROBP | Protein |
| | anhydrase | | | | cytotoxicity | | tyrosine |
| | | | | | triggering | | kinase binding |
| | | | | | receptor | | protein |
| CARD9 | Caspase | HOXA9 | Homeobox protein A9 | NEIL1 | nei | VEGFA | Vascular |
| | recruitment | | | | endonuclease | | endothelial |
| | domain 9 | | | | VIII-like 1 | | growth factor |
| | | | | | | | A |
| CASP1 | Caspase | HOXB3 | Homeobox B3 | NEIL2 | nei | WEE1 | WEE1 |
| | | | | | endonuclease | | homolog |
| | | | | | VIII-like 2 | | |
| CAT | Catalase | HOXB4 | Homeobox B4 | NFAT5 | Nuclear factor of | ZAP70 | Zeta chain |
| | | | | | activated T-cells | | (TCR) |
| | | | | | | | associated |
| | | | | | | | protein kinase |
| CCL3 | Chemokine | HPRT1 | Hypoxanthine | NFATC1 | Nuclear factor of | ZNF516 | Zinc finger |
| | receptor 3 | | phosphoribosyl- | | activated T- | | protein 516 |
| | | | transferase | | cells, | | |
| | | | | | cytoplasmic, | | |
| | | | | | calcineurin | | |
| | | | | | dependent | | |
| CCL4 | Chemokine | HRH1 | Histamine receptor | NFATC2 | Nuclear factor of | CSF2 | Colony |
| | receptor 4 | | | | activated T- | | stimulating |
| | | | | | cells, | | factor 2 |
| | | | | | cytoplasmic | | |
| CCL5 | T-cell specific | HRH2 | Histamine receptor | NOS2 | Nitric oxide | CSNK2A1 | Casein kinase |
| | protein | | | | synthase | | 2 |
| CCNB1 | Cyclin B1 | ICOS | Inducible T-cell | NOTCH1 | Notch homolog | CTGF | Connective |
| | | | costimulator | | 1, translocation | | tissue growth |
| | | | | | associated | | factor |
| CCND1 | BCL1 | ICOSLG | Inducible T-cell | NR3C1 | Nuclear receptor | CTLA4 | Cytotoxic T- |
| | oncogene | | costimulator ligand | | subfamily | | lymphocyte |
| | | | | | | | associated |
| | | | | | | | protein |
| CCR1 | Chemokine | ID2 | Inhibitor of DNA | NR4A1 | Nuclear receptor | CTNNA1 | Catenin |
| | receptor 1 | | binding | | subfamily | | (cadherin- |
| | | | | | | | associated |
| | | | | | | | protein) alpha-1 |
| CCR2 | Chemokine | IDO1 | Indoleamine-pyrrole | NRIP1 | Nuclear receptor | CTNNB1 | Catenin |
| | receptor 2 | | dioxygenase | | interacting | | cadherin |
| | | | | | protein | | associated |
| | | | | | | | protein |
| CCR4 | Chemokine | IFNA1 | Interferon alpha 1 | NT5E | 5-nucelotidase | CTNNBL1 | Beta catenin |
| | receptor 4 | | | | | | like protein |
| CCR5 | Chemokine | IFNG | Interferon gamma | OPTN | Tumor necrosis | CX3CL1 | Chemokine |
| | receptor 5 | | | | factor alpha | | ligand 1 |
| | | | | | inducible cellular | | |
| | | | | | protein | | |
| | | | | | containing | | |
| | | | | | leucine zipper | | |
| | | | | | domains | | |
| CCR6 | Chemokine | IFNGR1 | Interferon gamma | P2RX7 | Purinergic | CX3CR1 | Chemokine |
| | receptor 6 | | receptor | | receptor | | (C-X3-C motif) |
| | | | | | | | receptor 1 |
| CCR6 | Chemokine | IGF1R | Insulin like growth | PAX5 | B-cell specific | CXCL10 | Chemokine |
| | receptor 6 | | factor 1 receptor | | transcription | | ligand 10 |
| | | | | | factor | | |
| CCR7 | Chemokine | IKZF1 | IKAROS family zinc | PDCD1 | Programmed | CXCL12 | Pre-B cell |
| | receptor 7 | | finger 1 | | cell death | | growth |
| | | | | | protein | | stimulating |
| | | | | | | | factor |
| CD160 | NK cell | IL10 | Interleukin 10 | PDCD1LG2 | Programmed | CXCR3 | Chemokine |
| | receptor, | | | | cell death 1 | | receptor |
| | immunoglobulin | | | | ligand 2 | | |
| | superfamily | | | | | | |
| | member | | | | | | |
| CD19 | B-lymphocyte | IL10RA | Interleukin 10 receptor | PDCD1LG2 | Programmed | CXCR4 | Chemokine |
| | surface | | alpha | | cell death ligand | | (C-X3-C motif) |
| | antigen | | | | | | receptor 4 |
| CD2 | CD2 antigen | IL12A | Interleukin 12 alpha | PDE3A | Phospho- | DPP4 | Dipeptidyl |
| | | | | | diesterase 3A | | peptidase |
| CD226 | CD226 antigen | IL12B | Interleukin 12 beta | PDE4A | Phospho- | EGLN1 | Proxyl |
| | | | | | diesterase | | hydroxylase |
| | | | | | cAMP specific | | domain |
| | | | | | | | containing |
| | | | | | | | protein |
| CD244 | NK cell | IL12RB1 | Interleukin 12 receptor | PDE7A | Phospho- | EGLN3 | EGL nine |
| | receptor | | beta 1 | | diesterase 7A | | homolog 3 |
| CD27 | T-cell | IL12RB2 | Interleukin 12 receptor | PDK1 | Pyruvate | ITGA5 | Integrin |
| | activation | | beta 2 | | dehydrogenase | | alpha 5 |
| | antigen | | | | kinase, isozyme | | |
| CD274 | Programmed | IL13 | Interleukin 13 | PDXK | Pyridoxal kinase | ITGAL | Leukocyte |
| | cell death | | | | | | function |
| | ligand | | | | | | associated |
| | | | | | | | molecule |
| CD276 | Costimulatory | IL15 | Interleukin 15 | PECAM1 | Platelet | ITGAM | Neutrophil |
| | molecule | | | | endothelial cell | | adherence |
| | | | | | adhemsion | | receptor |
| | | | | | molecule 1 | | |
| CD28 | CD28 antigen | IL15RA | Interleukin 15 receptor | PHACTR2 | Phosphatase | ITGB1 | Fibronectin |
| | | | alpha | | and actin | | receptor |
| | | | | | regulator | | subunit beta |
| CD300A | Leukocyte | IL17A | Interleukin 17 alpha | PHC1 | Early | ITK | IL2 inducible |
| | membrane | | | | development | | T-cell kinase |
| | antigen | | | | regulator | | |
| CD38 | CD38 antigen | IL17F | Interleukin 17F | POP5 | Processing of | JAK1 | Tyrosine |
| | | | | | precursor | | protein kinase |
| CD3D | OKT3 delta | IL17RA | Interleukin 17 receptor | PPARA | Peroxisome | JAK2 | Janus kinase |
| | chain | | alpha | | proliferator- | | 2 |
| | | | | | activated | | |
| | | | | | receptor alpha | | |
| CD3E | T-cell surface | IL18 | Interleukin 18 | PPP2R1A | Protein | JAK3 | Leukocyte |
| | antigen | | | | phosphatase | | janus kinase |
| CD40LG | CD40 ligand | IL18R1 | Interleukin 18 receptor 1 | PRDM1 | Positive | JUN | Enhancer |
| | | | | | regulatory | | binding protein |
| | | | | | domain binding | | |
| | | | | | factor | | |
| CD44 | Phagocytic | IL18RAP | Receptor accessory | PRF1 | Perforin | JUNB | June B proto |
| | glycoprotein | | protein | | | | oncogene |
| CD47 | Leukocyte | IL1A | Interleukin 1 alpha | PROM1 | Prominin 1 | KIT | Mast/stem cell |
| | surface | | | | | | growth factor |
| | antigen | | | | | | receptor |
| CD58 | Lymphocyte | IL1B | Interleukin 1 beta | PTGER2 | Prostaglandin E | KLF10 | Kruppel like |
| | function | | | | receptor 2 | | factor 10 |
| | associated | | | | | | |
| | antigen | | | | | | |
| CD63 | Melanoma | IL2 | Interleukin 2 | PTK2 | Protein tyrosine | KLF2 | Kruppel like |
| | associated | | | | kinase 2 | | factor 2 |
| | antigen | | | | | | |
| CD69 | Early T-cell | IL21R | Interleukin 21 receptor | PTPN11 | Protein tyrosine | KLF4 | Kruppel like |
| | activation | | | | phosphatase | | factor 4 |
| | antigen | | | | | | |
| CD80 | T/B | IL22 | Interleukin 22 | PTPRK | Protein tyrosine | KLF6 | Kruppel like |
| | lymphocyte | | | | phosphatase | | factor 6 |
| | activation | | | | | | |
| | antigen | | | | | | |
| CD86 | T-lymphocyte | IL23A | Interleukin 23 alpha | RAC1 | Migration | KLRB1 | Killer cell |
| | activation | | | | inducing gene | | lectin like |
| | antigen | | | | | | receptor |
| CD8A | T-lymphocyte | IL23R | Interleukin 23 receptor | RAF1 | Murine leukemia | SH2B3 | Lymphocyte |
| | differentiation | | | | viral oncogene | | adaptor |
| | antigen | | | | homolog | | protein |
| CDH1 | Cadherin 1 | IL2RA | Interleukin 2 receptor | RAP1GAP2 | RAP1 GTP-ase | SIT1 | Signaling |
| | | | alpha | | activating | | threshold |
| | | | | | protein 2 | | regulating |
| | | | | | | | transmembrane |
| | | | | | | | adaptor |
| CDK2 | Cyclin | IL2RB | Interleukin 2 receptor | RARA | Retinoic acid | SKAP2 | Src kinase |
| | dependent | | beta | | receptor, alpha | | associated |
| | kinase | | | | | | phosphoprotein |
| CDK4 | Cyclin | IL2RG | Interleukin 2 receptor | RHOA | Ras homolog | SLA2 | Modulation of |
| | dependent | | gamma | | gene family | | antigen |
| | kinase | | | | | | receptor |
| | | | | | | | signaling |
| CDKN1A | Cyclin | IL4 | Interleukin 4, B-cell | RORA | Retinoid related | SLAMF1 | Signaling |
| | dependent | | stimulatory factor | | orphan receptor | | lymphocytic |
| | kinase inhibitor | | | | alpha | | activation |
| | 1A | | | | | | molecule |
| CDKN1B | Cyclin | IL4R | Interleukin 4 receptor | RORC | Retinoid related | SLAMF7 | Signaling |
| | dependent | | | | orphan receptor | | lymphocytic |
| | kinase inhibitor | | | | gamma | | activation |
| | | | | | | | molecule |
| CDKN2C | Cyclin | IL5 | Interleukin 5 | RUNX2 | Acute myeloid | SLC2A1 | Glucose |
| | dependent | | | | leukemia protein | | transporter |
| | kinase inhibitor | | | | | | type 1 |
| CEBPA | Enhancer | IL6 | Interleukin 6 | S100A4 | Leukemia | SMAD3 | Mothers |
| | binding protein | | | | multidrug | | against |
| | | | | | resistance | | decapentaplegic |
| | | | | | protein | | homolog 3 |
| CFLAR | Caspase like | IL6R | Interleukin 6 receptor | SATB1 | Special AT rich | SNAI1 | Snail 1 zinc |
| | apoptosis | | | | sequence | | finger protein |
| | regulator | | | | binding protein | | |
| | protein | | | | | | |
| CIITA | MHC class II | IL7R | Interleukin 7 receptor | SCML1 | Sex comb on | SOD1 | Superoxide |
| | transactivator | | | | midleg | | dismutase |
| CITED2 | Melanocyte | IRF1 | Interferon regulator | SCML2 | Sex comb on | SPI1 | Spleen focus |
| | specific gene | | factor 1 | | midleg like | | forming virus |
| | | | | | protein | | |
| CLIC1 | Chloride | IRF2 | Interferon regulator | SEL1L | Suppressor of | STAT1 | Signal |
| | intracellular | | factor | | lin12 | | transducer |
| | channel | | | | | | and activator |
| | | | | | | | of transcription |
| CREB1 | cAMP | IRF4 | Interferon regulator | SELL | Selectin L | STAT4 | Signal |
| | responsive | | factor 4 | | | | transducer |
| | element | | | | | | and activator |
| | binding protein | | | | | | of transcription |
| CRIP1 | Cystein rich | ITGA1 | Integrin, alpha | SELPLG | Selectin P ligand | STAT5A | Signal |
| | protein | | | | | | transducer |
| | | | | | | | and activator |
| | | | | | | | of transcription |
| CSAD | Cysteine | ITGA4 | Integrin alpha 4 | SERPINE2 | Serpin | STAT5B | Signal |
| | sulfinic acid | | | | peptidase | | transducer |
| | decarboxylase | | | | inhibitor | | and activator |
| | | | | | | | of transcription |
| | | | | | | STAT6 | Signal |
| | | | | | | | transducer |
| | | | | | | | and activator |
| | | | | | | | of transcription, |
| | | | | | | | IL4 induced |

### Example 5

### Biomarkers

A. Granzyme B

The therapeutic potential of the infused T cells is dependent on their persistence and their effector function. T cells use granzyme B, which is up-regulated in activated CD8⁺ T cells, to mediate tumor cell lysis. Cells were stained for 30 minutes with mAbs at 4° C. in FACS buffer. Intracellular staining for granzyme B was undertaken on canine T cells fixed in BD Cytofix/Cytoperm solution (BD Biosciences) for 20 minutes at 4° C. After washing in BD perm wash buffer, cells were incubated with mouse anti-human granzyme B (BD Pharmingen, GB11, 560211) and isotype control (Mouse anti-Rat IgG2a, 558067, BD Pharmingen) for 30 minutes at 4° C. in 1:10 dilution in BD perm wash buffer.

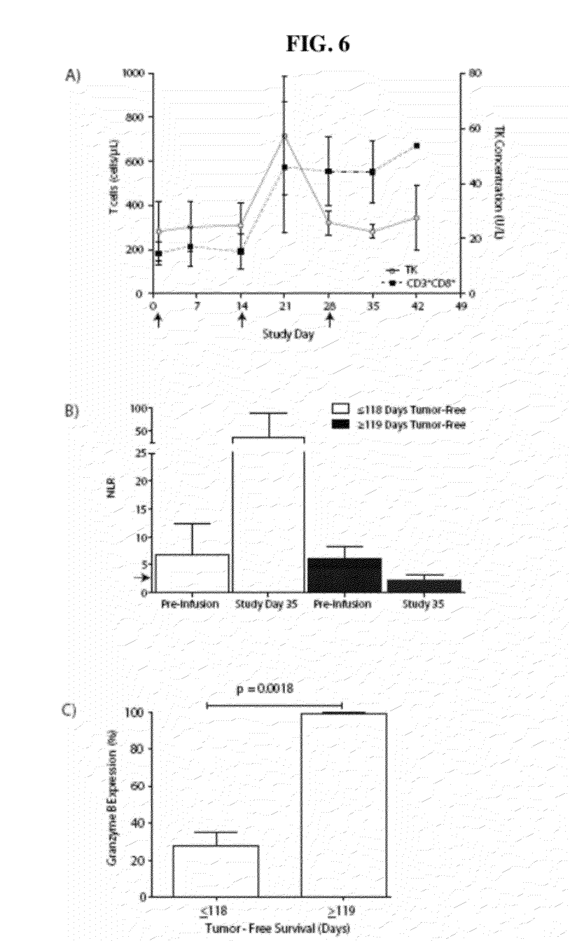

It was found that expression of granzyme B in the infusion product correlated with tumor-free survival (FIG. 6). In patients with shorter (≦118 days) tumor-free survival, the average granzyme B expression by the infusion product was 28±7%, compared to 99±0.5% in canines who were in remission for greater than 119 days. P42 achieved stable disease after infusion and was included in the tumor-free survival of less than 118 days. The correlation between the tumor-free survival and infusion product granzyme B was statistically significant (p=0.0018). These data demonstrate that inspection of the T-cell product may be used to stratify patients and their risk of progressive NHL disease.

B. Thymidine Kinase (TK)

TK is an enzyme that is elevated during DNA synthesis and the use of TK as predictor of infused T-cell persistence was tested. To determine whether TK was secreted by proliferating T cells, 10⁶ T cells from two donors were co-cultured for 48 hours with γ-irradiated OKT3-loaded CLN4 and cytokines resulting in a significantly elevated TK (28.6±2.5 U/L) in the supernatant compared to γ-irradiated and T cells co-cultured with cytokines only (p=0.03, 0.04). In particular, serum TK concentrations were serially assessed on collected serum using the Liaison TK kit (310960D, DiaSorin, Stillwater, Minn.). Tests were run according to manufacture's procedure on the Liaison analyzer (DiaSorin).

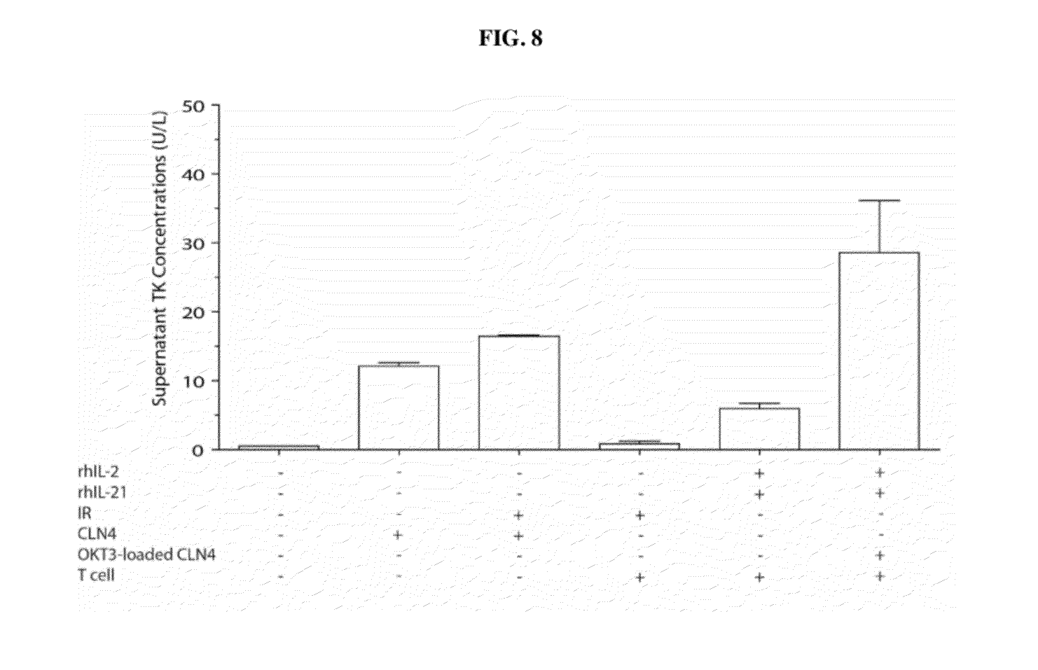

The source of TK appears to be the T cells as culturing them with cytokines alone also significantly increased TK concentrations (6.0±0.8) compared to γ-irradiated T cells (0.9±0.4, p=0.01) (FIG. 8). TK levels were significantly lower as measured from γ-irradiated or non-irradiated CLN4 (n=3), 16.4±0.2 U/L and 12.1±0.5 U/L, respectively. Increased TK can be attributed to T-cell lysis of CLN4 through the decreased CLN4 CD32⁺ expression and simultaneous increased NHL patient T-cell proliferation during propagation (FIG. 8). In vitro studies indicated that TK may be a useful biomarker for infused T-cell proliferation in response to T-cell receptor signaling.

Serum TK levels (FIG. 6) were elevated for 4 weeks after the T-cell infusions (study day 14: 24.8±8.3 U/L and study day 28: 49.2±18.5 U/L, n=5). The mean TK level decreased at study day 35 (21.4±2.1 U/L). These TK data appear to track with the presence of the adoptively transferred canine CD8⁺ T cells. The CD3⁺CD8⁺ population may have reached homeostasis after study day 28, resulting in the decrease of serum TK. Consistent from literature, serum from healthy canines (n=2) was analyzed and the mean TK concentration was 5.8±1.8 U/L, suggesting homeostasis and no inflammation.

C. Neutrophil to Lymphocyte Ratio (NLR)

As a further test for persistence of infused T cells, the ratio of lymphocytes to neutrophil counts was measured recognizing that ANC is a measurement of hematopoietic recovery after CHOP chemotherapy. An increased NLR has been shown to be a negative prognostic factor in human cancers, including NHL. When the infusion group was sorted into groups based on remission length and granzyme B expression, the overall ANC values through study 35 and the NLR decreased (p=0.04) in the patients with longer remission and greater granzyme B expression (FIG. 6). The pre-infusion NLR for patients in longer remission was 6:1±1.2 and it decreased significantly to 2.2:1±.5 by study day 35 (p=0.03). The pre-infusion NLR in shorter remission and stable disease patients was 6.7:1±3.2 and increased to 35.8:1±30.4 at study day 35, excluding the stable disease patient the NLR at study day 35 was 5.7:1±2. There was not a significant difference between study day 0 and study day 35 in patients with shorter remission lengths (p=0.3) excluding the stable disease patient.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A method of providing an anti-tumor response in a canine subject with cancer comprising infusing the subject with T cells.

2. The method of claim 1, wherein the T cells are ex-vivo propagated prior to said infusing.

3. The method of claim 1, wherein the T cells are propagated by culturing canine peripheral blood mononuclear cells with γ-irradiated artificial antigen presenting cells and a cytokine.

4. The method of claim 3, wherein the artificial antigen presenting cells are loaded with a CD3 antibody.

5. The method of claim 4, wherein the CD3 antibody is OKT3.

6. The method of claim 1, wherein the T cells are autologous T cells.

7. The method of claim 1, wherein the T cells comprise at least one marker selected from the group consisting of CD3⁺, CD4⁺, CD8⁺, CD25⁺, CD56⁺, CD21⁺ and CCR7⁺.

8. The method of claim 1, wherein the T cells are CD3⁺CD8⁺ cells.

9. The method of claim 1, wherein the T cells are CD3⁺CD4⁺ cells.

10. The method of claim 1, wherein the infusion is intravenous.

11. The method of claim 1, wherein the subject receives chemotherapy.

12. The method of claim 11, wherein the chemotherapy comprises cyclophosphamide, hydroxydaunorubicin (doxorubicin), Oncovin (vincristine), and prednisone/prednisolone.

13. The method of claim 11, wherein the subject receives chemotherapy prior to or during infusion of the T cells.

14. The method of claim 11, wherein the T cells are infused about 7 to about 488 days after completion of chemotherapy.

15. The method of claim 14, wherein the T cells are infused about 7 to about 21 days, about 98 to about 112 days or about 476 to about 488 days after the completion of chemotherapy.

16. The method of claim 1, wherein the subject is infused with about 5×10⁷/m² to about 3×10⁹ cells/m² T cells.

17. The method of claim 1, wherein the cancer is non-Hodgkin lymphoma.

18. A method for propagating canine T cells comprising culturing canine peripheral blood mononuclear cells with γ-irradiated artificial antigen presenting cells and a cytokine.

19. The method of claim 18, wherein the artificial antigen presenting cells are genetically modified to express T cell co-stimulatory ligands.

20. The method of claim 19, wherein the T cell co-stimulatory ligands are selected from the group consisting of CD19, CD64, CD86, CD137L, and membrane bound IL-15.

21. The method of claim 18, wherein the artificial antigen presenting cells are loaded with a CD3 antibody.

22. The method of claim 21, wherein the CD3 antibody is OKT3.

23. The method of claim 18, wherein the cytokine is an exogenous interleukin.

24. The method of claim 23, wherein the exogenous interleukin is IL-2 or IL-21.

25. The method of claim 23, wherein the exogenous interleukin is IL-2 and IL-21.

26. The method of claim 18, wherein the canine peripheral blood mononuclear cells are isolated from a canine with cancer.

27. The method of claim 26, wherein the cancer is non-Hodgkin lymphoma.

28. The method of claim 18, wherein the peripheral blood mononuclear cells are cultured with γ-irradiated artificial antigen presenting cells and a cytokine for up to 35 days.

29. The method of claim 28, wherein the peripheral blood mononuclear cells are cultured with γ-irradiated artificial antigen presenting cells and a cytokine for about 7, 14, 21, 28 or 35 days.

30. The method of claim 18, wherein the T cells comprise at least one marker selected from the group consisting of CD3⁺, CD4⁺, CD8⁺, CD25⁺, CD56⁺, CD21⁺ and CCR7⁺.

31. The method of claim 1, wherein the T cells are CD3⁺CD8⁺ cells.

32. The method of claim 1, wherein the T cells are CD3⁺CD4⁺ cells.
